(19) United States
(12) Patent Application Publication
(10) Pub. No.: US 2022/0273909 A1
(43) Pub. Date: Sep. 1, 2022

(54) FADE-OUT OF AUDIO TO MINIMIZE SLEEP DISTURBANCE FIELD

(71) Applicant: BOSE CORPORATION, Framingham, MA (US)

(72) Inventors: Harsh A. MANKODI, Brighton, MA (US); David Rolland CRIST, Watertown, MA (US); Chia-Chun HSU, Brighton, MA (US); Navaneethan SIVAGNANASUNDARAM, Waltham, MA (US); Chia-Ling LI, Framingham, MA (US); Kathleen Elizabeth KREMER, Southborough, MA (US)

(73) Assignee: BOSE CORPORATION, Framingham, MA (US)

(21) Appl. No.: 17/637,646

(22) PCT Filed: Aug. 5, 2020
(86) PCT No.: PCT/US20/44938

§ 371 Date: Feb. 23, 2022

Related U.S. Application Data
(60) Provisional application No. 62/890,960, filed on Aug. 23, 2019.

(51) Int. Cl. A61M 21/02; H04R 1/10; H04R 3/00; A61B 5/024; A61B 5/00

(52) U.S. Cl. CPC A61M 21/02; H04R 1/1083; H04R 3/00; A61B 5/02405; A61B 5/02438; A61B 5/681; A61M 2021/0027

(57) ABSTRACT

Aspects of the present disclosure provide methods, apparatuses, and systems for non-linearly decreasing an auditory experience output. According to an aspect, a non-linear decreasing rate is applied to an audio output of the auditory experience. The non-linear decreasing rate varies as a function of decibel amplitude over time in seconds. The non-linear decreasing rate comprises a plurality of segments connected together. The audio of the guided breathing is output at the non-linear decreasing rate until a decibel level of the audio output is below one of a decibel level of ambient noises in a user's environment or a predetermined decibel level.

Aspects of the present disclosure generally relate to methods, apparatuses, and systems for non-linearly decreasing guided breathing output.

### BACKGROUND

Utilizing guided breathing to regulate a user or subject's breathing rate, or amount of breaths taken per minute, can be beneficial in a number of health fields. For example, guided breathing can be used in several clinical applications, potentially leading to more effective or quicker treatments of conditions, including: asthma, stress, anxiety, insomnia, panic disorder, recurrent abdominal pain, chronic obstructive pulmonary disease, chronic hyperventilation, hypertension, and congestive heart failure, among others. Guided breathing may also be utilized to assist people in falling asleep and for meditation or relaxation purposes.

Many guided breathing exercises end by decreasing the output guided breathing by linearly decreasing the decibel amplitude over a period of time. However, linearly decreasing the decibel amplitude of the guided breathing over a period of time can cause the guided breathing to go too quiet or silent too quickly, which can be jarring to a user. In some cases, the guided breathing going silent too quickly may wake the user or disrupt the user's state of relaxation. Therefore, there is a need for outputting guided breathing exercises that is less noticeable to a user and that minimizes the chance of disrupting the user's sleep or state of relaxation.

### SUMMARY

Aspects of the present disclosure provide methods, apparatuses, and systems for non-linearly decreasing an auditory experience output. According to an aspect, a non-linear decreasing rate is applied to an audio output of the auditory experience. The non-linear decreasing rate varies as a function of decibel amplitude over time in seconds. The non-linear decreasing rate comprises a plurality of segments connected together. The audio of the guided breathing is output at the non-linear decreasing rate until a decibel level of the audio output is below one of a decibel level of ambient noises in a user's environment or a predetermined decibel level.

In an aspect, a method for outputting an auditory experience comprises applying a non-linear decreasing rate to an audio output of the auditory experience, and outputting the audio at the non-linear decreasing rate until a decibel level of the audio output is below one of a decibel level of ambient noises in a user's environment or a predetermined decibel level.

In an aspect, the non-linear decreasing rate is applied to one of an end or a transition of the auditory experience, and wherein the auditory experience is selected from the group consisting of guided breathing, masking noises, and binaural beats.

In an aspect, the non-linear decreasing rate varies as a function of decibel amplitude over time in seconds. The non-linear decreasing rate comprises a plurality of segments, wherein at least two of the plurality of segments has a different slope. The plurality of segments comprises a first segment, a second segment, and a third segment, the first segment having a first slope, the second segment having a second slope, and the third segment having a third slope, wherein the second slope is greater than the first slope, and wherein the third slope is greater than the second slope of the second segment. The plurality of segments comprise one or more segments having a first slope that decreases the decibel amplitude over the time in seconds, and one or more segments having a second slope that has a constant decibel amplitude over the time in seconds. The plurality of segments comprises a first segment, a second segment, a third segment, a fourth segment, a fifth segment, the first segment having the first slope, the second segment having the second slope connected to the first segment, the third segment having the first slope connected to the second segment, the fourth segment having the second slope connected to the third segment, and the fifth segment having the first slope connected to the fourth segment.

In an aspect, a stimulus output system comprises at least one transducer configured to output an auditory experience to a user, and a processor, the processor configured to output the auditory experience by applying a non-linear decreasing rate to an audio output of the auditory experience, and outputting the audio at the non-linear decreasing rate until a decibel level of the audio output is below one of a decibel level of ambient noises in a user's environment or a predetermined decibel level.

In an aspect, the non-linear decreasing rate is applied to one of an end or a transition of the auditory experience, and wherein the auditory experience is selected from the group consisting of guided breathing, masking noises, and binaural beats.

In an aspect, the non-linear decreasing rate varies as a function of decibel amplitude over time in seconds. The non-linear decreasing rate comprises a plurality of segments, wherein at least two of the plurality of segments has a different slope. The plurality of segments comprises a first segment, a second segment, and a third segment, the first segment having a first slope, the second segment having a second slope, and the third segment having a third slope, wherein the second slope is greater than the first slope, and wherein the third slope is greater than the second slope of the second segment. The plurality of segments comprise one or more segments having a first slope that decreases the decibel amplitude over the time in seconds, and one or more segments having a second slope that has a constant decibel amplitude over the time in seconds. The plurality of segments comprises a first segment, a second segment, a third segment, a fourth segment, a fifth segment, the first segment having the first slope, the second segment having the second slope connected to the first segment, the third segment having the first slope connected to the second segment, the fourth segment having the second slope connected to the third segment, and the fifth segment having the first slope connected to the fourth segment.

In an aspect, a wearable audio device comprises at least one speaker configured to output an auditory experience to a user, and a processor, the processor configured to output the auditory experience by applying a non-linear decreasing rate to an audio output of auditory experience, and outputting the audio at the non-linear decreasing rate until a decibel level of the audio output is below one of a decibel level of ambient noises in a user's environment or a predetermined decibel level.

In an aspect, the non-linear decreasing rate is applied to one of an end or a transition of the auditory experience, and wherein the auditory experience is selected from the group consisting of guided breathing, masking noises, and binaural beats.

In an aspect, the non-linear decreasing rate varies as a function of decibel amplitude over time in seconds. The non-linear decreasing rate comprises a plurality of segments, wherein at least two of the plurality of segments has a different slope. The plurality of segments comprises a first segment, a second segment, and a third segment, the first segment having a first slope, the second segment having a second slope, and the third segment having a third slope, wherein the second slope is greater than the first slope, and wherein the third slope is greater than the second slope of the second segment. The plurality of segments comprise one or more segments having a first slope that decreases the decibel amplitude over the time in seconds, and one or more segments having a second slope that has a constant decibel amplitude over the time in seconds. The plurality of segments comprises a first segment, a second segment, a third segment, a fourth segment, a fifth segment, the first segment having the first slope, the second segment having the second slope connected to the first segment, the third segment having the first slope connected to the second segment, the fourth segment having the second slope connected to the third segment, and the fifth segment having the first slope connected to the fourth segment.

All examples and features mentioned herein can be combined in any technically possible manner

### BRIEF DESCRIPTION OF THE DRAWINGS

FIG. 1 illustrates an example stimulus output system in a sleeping environment.

FIG. 2 illustrates example components of a stimulus output device.

FIG. 3 illustrates an example graph of a first non-linear decreasing rate and a second non-linear decreasing rate as a function of decibel (dB) amplitude over a period of time in seconds.

FIG. 4 illustrates an example graph of the first non-linear decreasing rate and the second non-linear decreasing rate of FIG. 3 as a function of decibel (dB) amplitude over a period of time in seconds compared to conventional linear decreasing rates.

FIG. 5 illustrates an example graph of a third non-linear decreasing rate and a fourth non-linear decreasing rate as a function of decibel (dB) amplitude over a period of time in seconds.

FIG. 6 illustrates an example graph of the third non-linear decreasing rate and the fourth non-linear decreasing rate of FIG. 5 as a function of decibel (dB) amplitude over a period of time in seconds compared to conventional linear decreasing rates.

### DETAILED DESCRIPTION

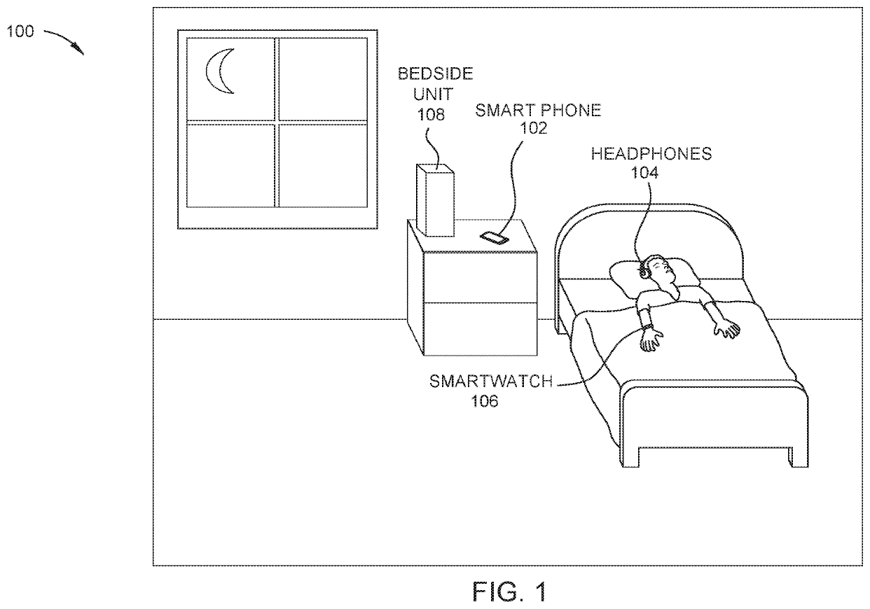

FIG. 1 illustrates an example stimulus output system 100 in a sleeping environment, according to an aspect. The stimulus output system 100 may be used to apply a non-linear decreasing rate to the audio output of an auditory experience, such as guided breathing, masking noises, and/or binaural beats, and to output the audio at the non-linear decreasing rate until a decibel level of the audio output is below one of a decibel level of ambient noises in a user's environment or a predetermined decibel level. The stimulus output system 100 may be an audio system including any combination of components shown in FIG. 1 and described herein.

The stimulus output system 100 includes headphones 104 and a smartwatch 106, which are shown as being worn by a subject or user. A headphone 104 refers to a device that fits around, on, or in an ear and that radiates acoustic energy into the ear canal. Headphones 104 are sometimes referred to as earphones, earpieces, headsets, earbuds, or sport headphones, and can be wired or wireless. The headphones 104 may comprise one or more of: a processing unit, a transceiver, one or more biosensors, one or more speakers, one or more systems configured to output any combination of haptics, lighting and audio, and one or more microphones. The headphones 104 may comprise an interface configured to receive input from a subject or user. A smartwatch 106 may be any type of wearable computer designed to be worn on a wrist of a subject or user, such as a fitness tracker. The smartwatch 106 may comprise one or more of: a processing unit, a transceiver, one or more biosensors, one or more speakers, one or more haptic systems, and one or more microphones. The smartwatch 106 may comprise an interface configured to receive input from a subject or user.

The stimulus output system 100 further includes a bedside unit 108 and a smartphone 102. The smartphone 102 may be a mobile phone, tablet, phablet, or laptop computer. The smartphone 102 may comprise one or more of: a processing unit, a transceiver, one or more biosensors, one or more speakers, one or more haptic systems, one or more light sources, and one or more microphones. The smartphone 102 may comprise an interface configured to receive input from a subject or user. The bedside unit 108 may be a stationary smart device, such as a smart speaker. The bedside unit 108 may have any shape and size capable of fitting on a surface in the sleeping environment, such as a dresser, desk, or night table. The bedside unit 108 may comprise one or more of: a processing unit, a transceiver, one or more biosensors, one or more speakers, one or more haptic systems, one or more light sources, and one or more microphones. In one aspect, the bedside unit 108 comprises one or more contactless biosensors, such as a radio frequency (RF) sensor, a radar sensor, or an under-bed accelerometer and/or microphone. The bedside unit 108 may comprise an interface configured to receive input from a subject or user.

The headphones 104, the smartwatch 106, the bedside unit 108, and the smartphone 102 may each include any wired or wireless communication means suitable for use with any other device 102-108 disposed in the sleeping environment, such as WiFi, Bluetooth, Near Field Communications (NFC), USB, micro USB, or any suitable wired or wireless communications technologies known to one of ordinary skill in the art. For example, the headphones 104 may comprise one or more speakers while the bedside unit 108 comprises one or more biosensors in communication with the one or more speakers of the headphones 104. Furthermore, the stimulus output system 100 may include one or more of the devices 102-108, and is not required to include each device 102-108 shown. Thus, each device 102-108 in the stimulus output system 100 may be optionally included, and only one device 102-108 is needed to output an auditory experience, such as guided breathing, masking noises, and/or binaural beats, and to non-linearly decrease the auditory experience output.

The devices 102-108 of the stimulus output system 100, either alone or in combination, are configured to: output an auditory experience, such as guided breathing, masking noises, and/or binaural beats, apply a non-linear decreasing rate to the audio output of the auditory experience, and to output the audio at the non-linear decreasing rate until a decibel level of the audio output is below one of a decibel level of ambient noises in a user's environment or a predetermined decibel level. The stimulus output system 100 may output a guided breathing stimulus to a user in the form of audio, haptics, lights, etc.

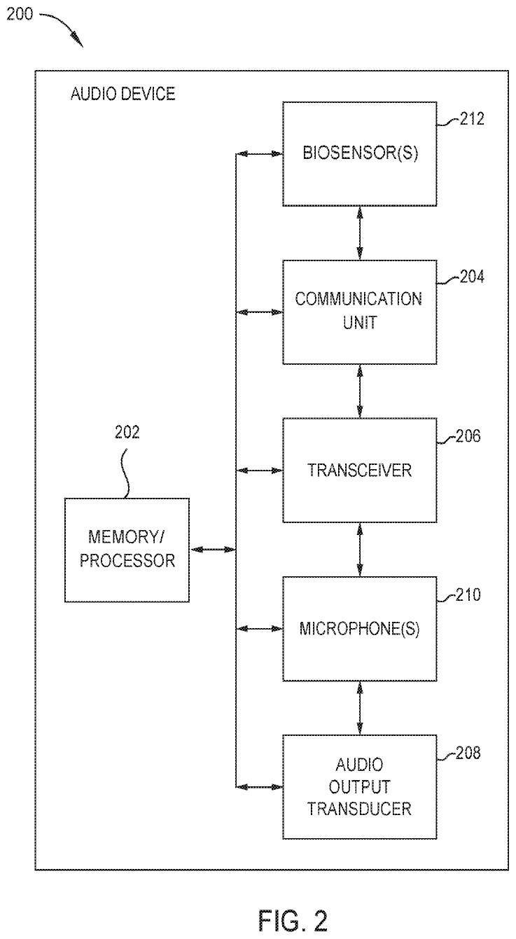

FIG. 2 illustrates example components of a stimulus output device 200, in accordance with certain aspects of the present disclosure. According to an example, the stimulus output device 200 is a wireless wearable audio device. The stimulus output device 200 may be an audio output device. The stimulus output device 200 may be used in a stimulus output system, such as the stimulus output system 100 of FIG. 1. For instance, the stimulus output device 200 may be any device 102-108 in the stimulus output system 100 of FIG. 1. In one example, the stimulus output device 200 is the headphones 104 of FIG. 1. In another example, the stimulus output device 200 is the bedside unit 108 of FIG. 1. The stimulus output device 200 may be used to apply a non-linear decreasing rate to the audio output of an auditory experience, such as guided breathing, masking noises, and/or binaural beats, and to output the audio at the non-linear decreasing rate until a decibel level of the audio output is below one of a decibel level of ambient noises in a user's environment or a predetermined decibel level.

The stimulus output device 200 includes a memory and processor 202, communication unit 204, a transceiver 206, a biosensor 212, and a speaker or audio output transducer 208. The memory may include Read Only Memory (ROM), a Random Access Memory (RAM), and/or a flash ROM. The memory stores program code for controlling the memory and processor 202. The memory and processor 202 control the operations of the stimulus output device 200. Any or all of the components in FIG. 2 may be combined into multi-function components.

The processor 202 controls the general operation of the stimulus output device 200. For example, the processor 202 performs process and control for audio and/or data communication. The processor 202 is configured to apply a non-linear decreasing rate to the audio output of an auditory experience, such as guided breathing, masking noises, and/or binaural beats. The processor 202 is configured to measure, receive, calculate, or detect at least one biosignal parameter of the subject. In combination with the audio output transducer 208, the processor 202 is configured to output audio at the non-linear decreasing rate until a decibel level of the audio output is below one of a decibel level of ambient noises in a user's environment or a predetermined decibel level. The processor 202 may be further configured to receive input from a subject or user, such as input regarding a predetermined decibel level at which the guided stimulus or auditory experience should cease being output. In at least one example, the processor 202 is disposed on another device in an audio system, such as a smartphone, and is in communication with the stimulus output device 200.

The communication unit 204 facilitates a wireless connection with one or more other wireless devices, such as with other devices in an audio system. For example, the communication unit 204 may include one or more wireless protocol engines such as a Bluetooth engine. While Bluetooth is used as an example protocol, other communication protocols may also be used. Some examples include Bluetooth Low Energy (BLE), NFC, IEEE 802.11, WiFi, or other local area network (LAN) or personal area network (PAN) protocols. The stimulus output device 200 may receive audio files wirelessly via the communication unit 204. Additionally or alternatively, the communication unit 204 may receive information associated with a subject's biosignal parameters, obtained via a contactless sensor. Examples of contactless sensors include a radio frequency (RF) sensor, a radar sensor, or an under-bed accelerometer.

The transceiver 206 transmits and receives information via one or more antennae to exchange information with one or more other wireless devices. The transceiver 206 may be used to communicate with other devices in an audio system, such as a bedside unit, a smartphone, and/or a smartwatch. The transceiver 206 is not necessarily a distinct component.

The stimulus output device 200 includes the audio output transducer 208, which may be also known as a driver or speaker. In some examples, more than one output transducer 208 is used. The transducer 208 (that may be part of a microphone) converts electrical signals into sound and converts sound into electrical signals. The transducer 208 is configured to output a guiding stimulus to a user or subject. The transducer 208 outputs audio signals, including adjusted audio signals in an effort to regulate a user's breathing. For example, the transducer 208 may be configured to adjust audio signals in response to a subject's biosignal parameters. In at least one example, the transducer 208 is disposed on another device in an audio system, such as a bedside unit, and is in communication with the stimulus output device 200.

The stimulus output device 200 optionally includes one or more microphones 210. In an aspect, the microphones 210 are used to convert noises into electrical signals. In at least one example, one or more microphones 210 are disposed on another device in an audio system, such as a bedside unit, and are in communication with the stimulus output device 200. The microphone 210 may be used to approximate or measure the decibel level of the ambient noise in the user's environment.

The stimulus output device 200 optionally includes one or more biosensors 212 used to determine, sense, measure, monitor, or calculate a biosignal parameter of a subject wearing the stimulus output device 200.

According to an aspect when the stimulus output device 200 is headphones, only one earpiece (ear tip, ear cup) of the stimulus output device 200 includes the biosensor 212. In an aspect, neither earpiece includes a biosensor 212. Instead, a biosensor 212, not on the stimulus output device 200, may remotely detect a biosignal parameter of the subject. In an example, the biosensor 212 detects a subject's heartrate or heart rate variability (HRV) with a sensor disposed on the wrist, such as by utilizing a smartwatch. In an example, the biosensor 212 may be a contactless biosensor. The contactless biosensor is configured to report detected biosignal parameters to the processor 202, for example, via the communication unit 204. In at least one example, the biosensor 212 is disposed on another device in an audio system, such as a smartwatch, and is in communication with the stimulus output device 200.

FIG. 2 illustrates communication between certain modules of an example stimulus output device 200; however, aspects of the disclosure are not limited to the specific illustrated example. According to aspects, any module 202-212 is configured to communicate with any other module in the stimulus output device 200. In one example, all modules 202-212 are connected to and communicate with each other. The stimulus output device 200 may output a guided breathing stimulus to a user in the form of audio, haptics, lights, etc.

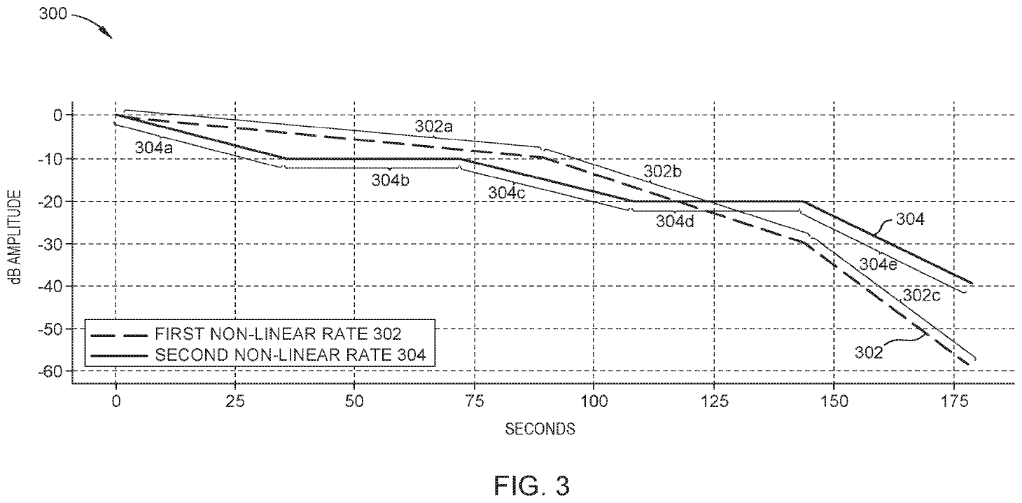

FIG. 3 illustrates an example graph 300 of a first non-linear decreasing rate 302 and a second non-linear decreasing rate 304 as a function of decibel (dB) amplitude over a period of time in seconds, according to aspects disclosed herein. The first and second non-linear decreasing rates 302, 304 may be applied to an audio output of an auditory experience, such as guided breathing, masking noises, and/or binaural beats. Specifically, the first and second non-linear decreasing rates 302, 304 may be applied to one of an end or a transition of the guided breathing, masking noises, and/or binaural beats. The first and second non-linear decreasing rates 302, 304 may each individually be utilized with the stimulus output system 100 of FIG. 1 and/or the stimulus output device 200 of FIG. 2.

The first non-linear decreasing rate 302 comprises a plurality of segments 302a-302c connected together. Each segment 302a-302c may be linear but connected together in such a way that the segments 302a-302c taken as a whole are non-linear. As shown in FIG. 3, the first non-linear decreasing rate 302 comprises a first segment 302a, a second segment 302b connected to the first segment 302a, and a third segment 302c connected to the second segment 302b. While three segments 302a-302c are shown, the first non-linear decreasing rate 302 may comprise any suitable number of segments (i.e., additional or fewer segments).

The first segment 302a of the first non-linear decreasing rate 302 has a first slope, the second segment 302b of the first non-linear decreasing rate 302 has a second slope different than the first slope, and the third segment 302c of the first non-linear decreasing rate 302 has a third slope different than the first and second slopes. The second slope of the second segment 302b is greater than or steeper than the first slope of the first segment 302a (i.e., the first segment 302a decreases about 10 dB while the second segment 302b decreases about 20 dB). The third slope of the third segment 302c is greater than or steeper than the second slope of the second segment 302b (i.e., the second segment 302b decreases about 20 dB while the third segment 302c decreases about 30 dB). As such, the slope of each segment 302a-302c increases as time progresses. Thus, when the first non-linear decreasing rate 302 is applied to the audio output of the guided breathing, the dB amplitude gradually decreases, and progressively decreases over time.

In one example, the first segment 302a of the first non-linear decreasing rate 302 may be applied for a longer amount of time than the second segment 302b of the first non-linear decreasing rate 302 and the third segment 302c of the first non-linear decreasing rate 302. Similarly, the second segment 302b may be applied for a longer amount of time than the third segment 302c. As shown in FIG. 3, the first segment 302a lasts for about 90 seconds, the second segment 302b lasts for about 50 seconds, and the third segment 302c lasts for about 40 seconds. Thus, the third segment 302c of the first non-linear decreasing rate 302 having the steepest slope may be applied for the shortest amount of time and the first segment 302a of the first non-linear decreasing rate 302 having the most gradual slope may be applied for the greatest amount of time. In another example, each segment 302a-302c of the first non-linear decreasing rate 302 may be applied for substantially the same amount of time.

The second non-linear decreasing rate 304 comprises a plurality of segments 304a-304e connected together. Each segment 304a-304e may be linear but connected together in such a way that the segments 304a-304e taken as a whole are non-linear. As shown in FIG. 3, the second non-linear decreasing rate 304 comprises a first segment 304a, a second segment 304b connected to the first segment 304a, a third segment 304c connected to the second segment 304b, a fourth segment 304d connected to the third segment 304c, and a fifth segment 304e connected to the fourth segment 304d. While five segments 304a-304e are shown, the second non-linear decreasing rate 304 may comprise any suitable number of segments (i.e., additional or fewer segments).

The first segment 304a of the second non-linear decreasing rate 304 has a first slope, the second segment 304b of the second non-linear decreasing rate 304 has a second slope different than the first slope, the third segment 304c of the second non-linear decreasing rate 304 has a third slope different than at least the second slope, the fourth segment 304d of the second non-linear decreasing rate 304 has a fourth slope different than at least the first and third slopes, and the fifth segment 304e of the second non-linear decreasing rate 304 has a fifth slope different than at least the second and fourth slopes. The second slope of the second segment 304b and the fourth slope of the fourth segment 304d may be the same and each have a constant decibel amplitude over the time in seconds. As shown in FIG. 3, the second slope of the second segment 304b is held constant at about −10 dB for about 30 seconds to about 40 seconds while the fourth slope of the fourth segment 304d is held constant at about −20 dB for about 30 seconds to about 40 seconds.

The first, third, and fifth slopes of the first, third, and fifth segments 304a, 304c, 304e may be the same or may be different. For example, the first slope of the first segment 304a may be the same as the third slope of the third segment 304c, but may be different than the fifth slope of the fifth segment 304e. In such an example, the fifth slope of the fifth segment 304e may be greater than or steeper than the first and third slopes of the first and third segments 304a, 304c. In another example, the first, third, and fifth slopes of the first, third, and fifth segments 304a, 304c, 304e are all substantially equal. In yet another example, the third slope of the third segment 304c is greater than or steeper than the first slope of the first segment 304a, and the fifth slope of the fifth segment 304e is greater than or steeper than the third slope of the third segment 304c.

In one aspect, each segment 304a-304e of the second non-linear decreasing rate 304 may be applied for substantially the same amount of time. In another aspect, each segment 304a-304e of the second non-linear decreasing rate 304 may be applied varying periods of time. For example, the first segment 304a may be applied for a longer amount of time than the second through fifth segments 304b-304e. In yet another aspect, the first, third, and fifth segments 304a, 304c, 304e may be applied for a first amount of time and the second and fourth segments 304b, 304d may be applied for a second amount of time different than the first amount of time. In such an example, the first amount of time may be greater than or less than the second amount of time.

In one aspect, the first and second non-linear decreasing rates 302, 304 may be applied to the audio output of the guided breathing or auditory experience until a dB level of the audio output is less than a dB level of ambient noises in the user's environment. The ambient noise level in the user's environment may be measured or approximated using a microphone, such as the microphone 210 of FIG. 2. In another aspect, the first and second non-linear decreasing rates 302, 304 may be applied to the audio output of the guided breathing or auditory experience until a predetermined dB level is reached. The predetermined dB level may be selected by a user or may be factory set. For example, the predetermined dB level may be an estimated or average ambient noise level. In another example, the predetermined dB level may be a preset dB level that is likely to be less than the ambient noise level in the user's environment, such as about −30 dB.

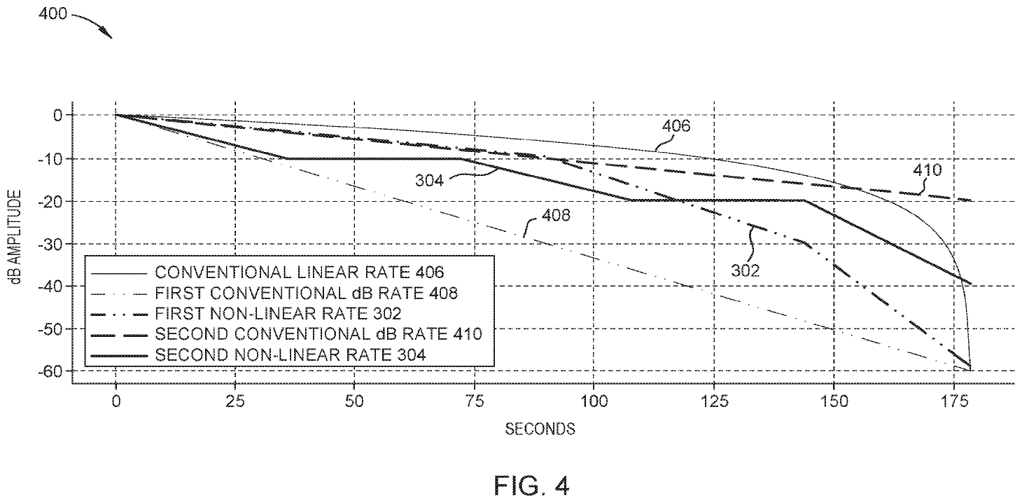

FIG. 4 illustrates an example graph 400 of the first non-linear decreasing rate 302 and the second non-linear decreasing rate 304 of FIG. 3 as a function of decibel (dB) amplitude over a period of time in seconds compared to conventional linear decreasing rates. For comparison purposes, the graph 400 shows a conventional linear amplitude rate 406, a first conventional linear dB amplitude rate 408, and a second conventional linear dB amplitude rate 410 to −20 dB as examples.

As shown in FIG. 4, the first and second non-linear decreasing rates 302, 304 gradually decrease the dB amplitude over time in a segmented or step-like manner, which is less noticeable to user than each of the conventional linear amplitude rate 406, the first conventional linear dB amplitude rate 408, and the second conventional linear dB amplitude rate 410. The conventional linear amplitude rate 406 has a drastic decrease or shift towards the end of the time period while the first conventional linear dB amplitude rate 408 has a drastic decrease or shift in the beginning of the time period. In other words, the conventional linear amplitude rate 406, the first conventional linear dB amplitude rate 408, and the second conventional linear dB amplitude rate 410 may go too quiet too quickly, alerting the user that the audio output of the auditory experience has ceased or transitioned.

As such, the conventional linear amplitude rate 406, the first conventional linear dB amplitude rate 408, and the second conventional linear dB amplitude rate 410 may each result in a sudden perceptual change that can be jarring or disturbing to a user, causing the user's sleep or state of relaxation to be interrupted. Conversely, the first and second non-linear decreasing rates 302, 304 steadily fade-out, making jarring or perceptual changes less likely, and providing for a smoother transition or end to the audio output of the guided breathing, masking noises, and/or binaural beats.

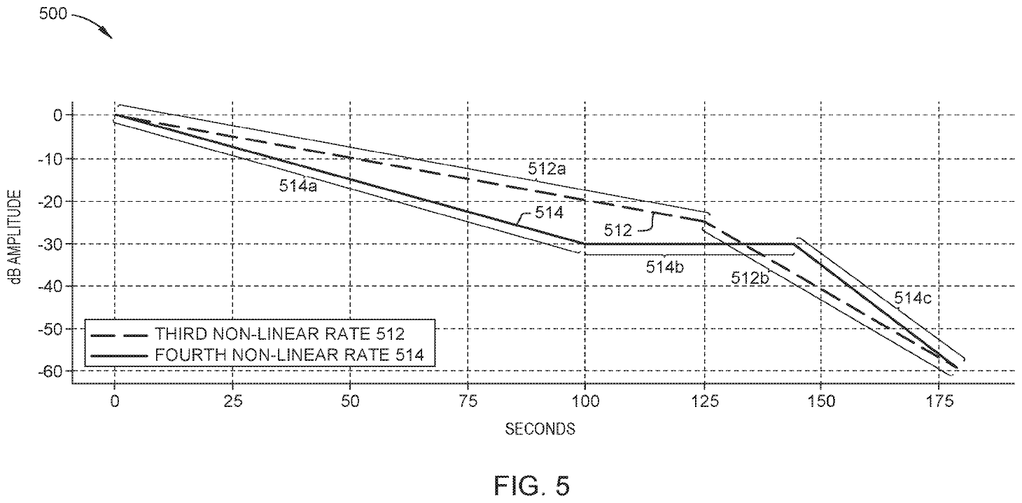

FIG. 5 illustrates an example graph 500 of a third non-linear decreasing rate 512 and a fourth non-linear decreasing rate 514 as a function of decibel (dB) amplitude over a period of time in seconds, according to aspects disclosed herein. The third and fourth non-linear decreasing rates 512, 514 may be applied to an audio output of an auditory experience, such as guided breathing, masking noises, and/or binaural beats. Specifically, the third and fourth non-linear decreasing rates 512, 514 may be applied to one of an end or a transition of the guided breathing, masking noises, and/or binaural beats. The third and fourth non-linear decreasing rates 512, 514 may each individually be utilized with the stimulus output system 100 of FIG. 1 and/or the stimulus output device 200 of FIG. 2. The third and fourth non-linear decreasing rates 512, 514 are similar to the first and second non-linear decreasing rates 302, 304; however, the third and fourth non-linear decreasing rates 512, 514 comprise fewer segments while achieving the same goal.

The third non-linear decreasing rate 512 comprises a plurality of segments 512a-512b connected together. Each segment 512a-512b may be linear but connected together in such a way that the segments 512a-512b taken as a whole are non-linear. As shown in FIG. 5, the third non-linear decreasing rate 512 comprises a first segment 512a and a second segment 512b connected to the first segment 512a. While two segments 512a-512b are shown, the third non-linear decreasing rate 512 may comprise any suitable number of segments (i.e., additional or fewer segments).

The first segment 512a of the third non-linear decreasing rate 512 has a first slope and the second segment 512b of the third non-linear decreasing rate 512 has a second slope different than the first slope. The second slope of the second segment 152b is greater than or steeper than the first slope of the first segment 512a (i.e., the first segment 512a decreases about 25 dB while the second segment 302b decreases about 35 dB). As such, the slope of each segment 512a-512b increases as time progresses. Thus, when the third non-linear decreasing rate 512 is applied to the audio output of the guided breathing, the dB amplitude gradually decreases, and progressively decreases over time.

In one example, the first segment 512a of the third non-linear decreasing rate 512 may be applied for a longer amount of time than the second segment 512b of the third non-linear decreasing rate 512. As shown in FIG. 5, the first segment 512a lasts for about 125 seconds and the second segment 512b lasts for about 50 seconds. In another example, each segment 512a-512b of the third non-linear decreasing rate 302 may be applied for substantially the same amount of time.

The fourth non-linear decreasing rate 514 comprises a plurality of segments 514a-514c connected together. Each segment 514a-514c may be linear but connected together in such a way that the segments 514a-514c taken as a whole are non-linear. As shown in FIG. 5, the fourth non-linear decreasing rate 514 comprises a first segment 514a, a second segment 514b connected to the first segment 514a, and a third segment 514c connected to the second segment 514b. While three segments 514a-514c are shown, the fourth non-linear decreasing rate 514 may comprise any suitable number of segments (i.e., additional or fewer segments).

The first segment 514a of the fourth non-linear decreasing rate 514 has a first slope, the second segment 514b of the fourth non-linear decreasing rate 514 has a second slope different than the first slope, and the third segment 514c of the fourth non-linear decreasing rate 514 has a third slope different than at least the second slope. The second slope of the second segment 514b has a constant decibel amplitude over the time in seconds. As shown in FIG. 5, the second slope of the second segment 514b is held constant at about −30 dB for about 40 seconds to about 50 seconds.

The first and third slopes of the first and third segments 514a, 514c may be the same or may be different. For example, the first slope of the first segment 514a may be different than the third slope of the third segment 514c. In such an example, the third slope of the third segment 514c may be greater than or steeper than the first slope of the first segment 514a. In another example, the first and third slopes of the first and third segments 514a, 514c are all substantially equal.

In one aspect, each segment 514a-514c of the fourth non-linear decreasing rate 514 may be applied for substantially the same amount of time. In another aspect, each segment 514a-514c of the fourth non-linear decreasing rate 514 may be applied varying periods of time. For example, the first segment 514a may be applied for a longer amount of time than the second and third segments 514b, 514c. In yet another aspect, the second segment 514c may be applied for a longer amount of time than the third segment 514c. Thus, when the fourth non-linear decreasing rate 514 is applied to the audio output of the guided breathing, the dB amplitude gradually decreases, and progressively decreases over time.

In one aspect, the third and fourth non-linear decreasing rates 512, 514 may be applied to the audio output of the guided breathing or auditory experience until a dB level of the audio output is less than a dB level of ambient noises in the user's environment. The ambient noise level in the user's environment may be measured or approximated using a microphone, such as the microphone 210 of FIG. 2. In another aspect, the third and fourth non-linear decreasing rates 512, 514 may be applied to the audio output of the guided breathing or auditory experience until a predetermined dB level is reached. The predetermined dB level may be selected by a user or may be factory set. For example, the predetermined dB level may be an estimated or average ambient noise level. In another example, the predetermined dB level may be a preset dB level that is likely to be less than the ambient noise level in the user's environment, such as about −30 dB.

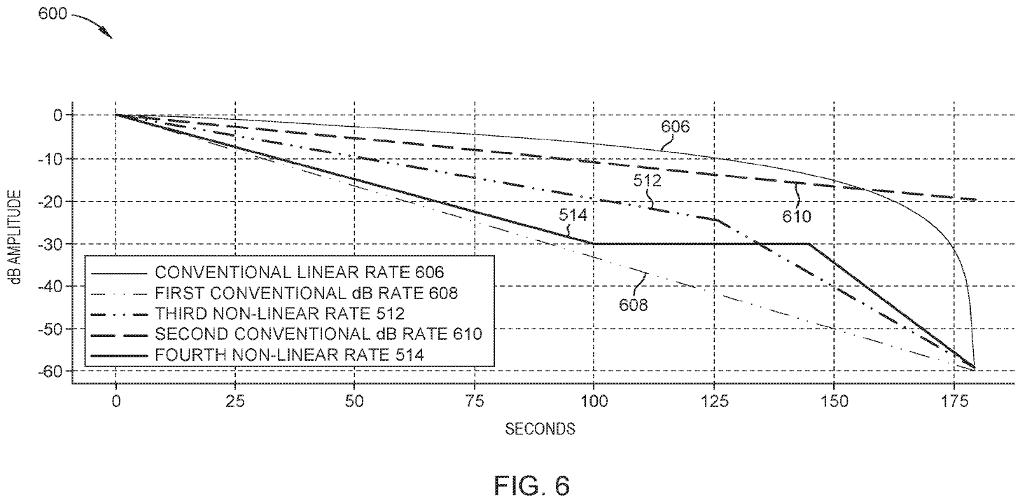

FIG. 6 illustrates an example graph 600 of the third non-linear decreasing rate 512 and the fourth non-linear decreasing rate 514 of FIG. 5 as a function of decibel (dB) amplitude over a period of time in seconds compared to conventional linear decreasing rates. For comparison purposes, the graph 600 shows a conventional linear amplitude rate 606, a first conventional linear dB amplitude rate 608, and a second conventional linear dB amplitude rate 610 to −20 dB as examples, similar to FIG. 4.

As shown in FIG. 6, the third and fourth non-linear decreasing rates 512, 514 gradually decrease the dB amplitude over time in a segmented or step-like manner, which is less noticeable to user than each of the conventional linear amplitude rate 606, the first conventional linear dB amplitude rate 608, and the second conventional linear dB amplitude rate 610. The conventional linear amplitude rate 606 has a drastic decrease or shift towards the end of the time period while the first conventional linear dB amplitude rate 608 has a drastic decrease or shift in the beginning of the time period. In other words, the conventional linear amplitude rate 606, the first conventional linear dB amplitude rate 608, and the second conventional linear dB amplitude rate 610 may go too quiet too quickly, alerting the user that the audio output of the auditory experience has ceased or transitioned.

As such, the conventional linear amplitude rate 606, the first conventional linear dB amplitude rate 608, and the second conventional linear dB amplitude rate 610 may each result in a sudden perceptual change that can be jarring or disturbing to a user, causing the user's sleep or state of relaxation to be interrupted. Conversely, the third and fourth non-linear decreasing rates 512, 514 steadily fade-out, making jarring or perceptual changes less likely, and providing for a smoother transition or end to the audio output of the guided breathing, masking noises, and/or binaural beats.

Therefore, by using a non-linear decreasing rate to subtly fade-out an audio output of an auditory experience, such as guided breathing, masking noises, and/or binaural beats, the end or transition of the auditory experience is less likely to be noticeable by a user or to disrupt a user's sleep or state of relaxation. As such, the non-linear decreasing rates minimize the chance that the auditory experience, which may have helped the user fall asleep or relax, is undone when the audio output fades out. Thus, applying the non-linear decreasing rate to the audio output of the auditory experience results in an overall more enjoyable and smoother experience for the user, as the user will be unaware the auditory experience has ceased or transitioned.

In the preceding, reference is made to aspects presented in this disclosure. However, the scope of the present disclosure is not limited to specific described aspects. Aspects of the present disclosure may take the form of an entirely hardware embodiment, an entirely software embodiment (including firmware, resident software, micro-code, etc.) or an embodiment combining software and hardware aspects that may all generally be referred to herein as a “component,” “circuit,” “module” or “system.” Furthermore, aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples of a computer readable storage medium include: an electrical connection having one or more wires, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the current context, a computer readable storage medium may be any tangible medium that can contain, or store a program.

The flowchart and block diagrams in the figures illustrate the architecture, functionality and operation of possible implementations of systems, methods and computer program products according to various aspects. In this regard, each block in the flowchart or block diagrams may represent a module, segment or portion of code, which comprises one or more executable instructions for implementing the specified logical function(s). In some implementations the functions noted in the block may occur out of the order noted in the figures. For example, two blocks shown in succession may, in fact, be executed substantially concurrently, or the blocks may sometimes be executed in the reverse order, depending upon the functionality involved. Each block of the block diagrams and/or flowchart illustrations, and combinations of blocks in the block diagrams and/or flowchart illustrations can be implemented by special-purpose hardware-based systems that perform the specified functions or acts, or combinations of special purpose hardware and computer instructions.

1. A method for outputting an auditory experience, comprising:
applying a non-linear decreasing rate to an audio output of the auditory experience; and
outputting the audio at the non-linear decreasing rate until a decibel level of the audio output is below one of a decibel level of ambient noises in a user's environment or a predetermined decibel level.

2. The method of claim 1, wherein the non-linear decreasing rate is applied to one of an end or a transition of the auditory experience, and wherein the auditory experience is selected from the group consisting of guided breathing, masking noises, and binaural beats.

3. The method of claim 1, wherein the non-linear decreasing rate varies as a function of decibel amplitude over time in seconds.

4. The method of claim 3, wherein the non-linear decreasing rate comprises a plurality of segments, wherein at least two of the plurality of segments has a different slope.

5. The method of claim 4, wherein the plurality of segments comprises a first segment, a second segment, and a third segment, the first segment having a first slope, the second segment having a second slope, and the third segment having a third slope, wherein the second slope is greater than the first slope, and wherein the third slope is greater than the second slope of the second segment.

6. The method of claim 4, wherein the plurality of segments comprise one or more segments having a first slope that decreases the decibel amplitude over the time in seconds, and one or more segments having a second slope that has a constant decibel amplitude over the time in seconds.

7. The method of claim 6, wherein the plurality of segments comprises a first segment, a second segment, a third segment, a fourth segment, a fifth segment, the first segment having the first slope, the second segment having the second slope connected to the first segment, the third segment having the first slope connected to the second segment, the fourth segment having the second slope connected to the third segment, and the fifth segment having the first slope connected to the fourth segment.

8. A stimulus output system, comprising:
at least one transducer configured to output an auditory experience to a user; and
a processor, the processor configured to output the auditory experience by:
applying a non-linear decreasing rate to an audio output of the auditory experience; and
outputting the audio at the non-linear decreasing rate until a decibel level of the audio output is below one of a decibel level of ambient noises in a user's environment or a predetermined decibel level.

9. The stimulus output system of claim 8, wherein the non-linear decreasing rate is applied to one of an end or a transition of the auditory experience, and wherein the auditory experience is selected from the group consisting of guided breathing, masking noises, and binaural beats.

10. The stimulus output system of claim 8, wherein the non-linear decreasing rate varies as a function of decibel amplitude over time in seconds.

11. The stimulus output system of claim 10, wherein the non-linear decreasing rate comprises a plurality of segments, wherein at least two of the plurality of segments has a different slope.

12. The stimulus output system of claim 11, wherein the plurality of segments comprises a first segment, a second segment, and a third segment, the first segment having a first slope, the second segment having a second slope, and the third segment having a third slope, wherein the second slope is greater than the first slope, and wherein the third slope is greater than the second slope of the second segment.

13. The stimulus output system of claim 11, wherein the plurality of segments comprise one or more segments having a first slope that decreases the decibel amplitude over the time in seconds, and one or more segments having a second slope that has a constant decibel amplitude over the time in seconds.

14. The stimulus output system of claim 13, wherein the plurality of segments comprises a first segment, a second segment, a third segment, a fourth segment, a fifth segment, the first segment having the first slope, the second segment having the second slope connected to the first segment, the third segment having the first slope connected to the second segment, the fourth segment having the second slope connected to the third segment, and the fifth segment having the first slope connected to the fourth segment.

15. A wearable audio device, comprising:
at least one speaker configured to output an auditory experience to a user; and
a processor, the processor configured to output the auditory experience by:
applying a non-linear decreasing rate to an audio output of auditory experience; and
outputting the audio at the non-linear decreasing rate until a decibel level of the audio output is below one of a decibel level of ambient noises in a user's environment or a predetermined decibel level.

16. The wearable audio device of claim 15, wherein the non-linear decreasing rate is applied to one of an end or a transition of the auditory experience, and wherein the auditory experience is selected from the group consisting of guided breathing, masking noises, and binaural beats.

17. The wearable audio device of claim 15, wherein the non-linear decreasing rate varies as a function of decibel amplitude over time in seconds.

18. The wearable audio device of claim 17, wherein the non-linear decreasing rate comprises a plurality of segments, wherein at least two of the plurality of segments has a different slope.

19. The wearable audio device of claim 18, wherein the plurality of segments comprises a first segment, a second segment, and a third segment, the first segment having a first slope, the second segment having a second slope, and the third segment having a third slope, wherein the second slope is greater than the first slope, and wherein the third slope is greater than the second slope of the second segment.

20. The wearable audio device of claim 18, wherein the plurality of segments comprise one or more segments having a first slope that decreases the decibel amplitude over the time in seconds, and one or more segments having a second slope that has a constant decibel amplitude over the time in seconds.

21. The wearable audio device of claim 20, wherein the plurality of segments comprises a first segment, a second segment, a third segment, a fourth segment, a fifth segment, the first segment having the first slope, the second segment having the second slope connected to the first segment, the third segment having the first slope connected to the second segment, the fourth segment having the second slope connected to the third segment, and the fifth segment having the first slope connected to the fourth segment.
